(19) United States
(12) Patent Application Publication
(10) Pub. No.: US 2016/0279049 A1
(43) Pub. Date: Sep. 29, 2016

(54) COSMETIC HAIR CLEANING COMPOSITION

(71) Applicant: Henkel AG & Co. KGaA, Duesseldorf (DE)

(72) Inventors: Thomas Schroeder, Hamburg (DE); Dirk Hentrich, Hamburg (DE); Corinna Bender, Hamburg (DE)

(73) Assignee: Henkel AG & Co. KGaA, Duesseldorf (DE)

(21) Appl. No.: 15/175,869

(22) Filed: Jun. 7, 2016

Related U.S. Application Data
(63) Continuation of application No. PCT/EP2014/074436, filed on Nov. 13, 2014.

(30) Foreign Application Priority Data
Dec. 17, 2013 (DE) 10 2013 226 269.8

(51) Int. Cl. A61K 8/73; A61K 8/42; A61Q 5/12; A61K 8/92; A61K 8/46; A61Q 5/02; A61K 8/891; A61K 8/86

(52) U.S. Cl. CPC A61K 8/737; A61K 8/891; A61K 8/42; A61K 8/86; A61K 8/922; A61K 8/463; A61Q 5/02; A61Q 5/12; A61K 2800/594; A61K 2800/5426; A61K 2800/49

(57) ABSTRACT

A cosmetic cleaning agent includes, in a cosmetic vehicle, a) at least one anionic and/or amphoteric (zwitterionic) surfactant, b) at least one cationic polysaccharide, c) at least one silicone, d) at least one wax, and e) at least one virgin oil and/or at least one cationic protein hydrolysate. The cosmetic cleaning agent may be used to eliminate, reduce, and/or avoid hair breakage.

### FIELD OF THE INVENTION

The present invention generally relates to cosmetics, and more particularly relates to rinse-out cleaning agents that are used to eliminate, reduce, and/or prevent hair breakage and a method for cleaning hair and/or for eliminating, reducing, and/or preventing hair breakage.

### BACKGROUND OF THE INVENTION

Cosmetic cleaning agents, such as hair shampoos, are usually based on traditional anionic, amphoteric, zwitterionic, non-ionic, and/or cationic surfactants. Because of their excellent cleaning and foaming ability, anionic surfactants are predominantly used, possibly in mixture with small amounts of co-surfactants. Such a commercially available shampoo cleans the hair and eliminates sebum residues and/or residues of styling agents and other soiling from the hair surface and the scalp. However, lipids and proteins are also removed from the hair and the scalp during the cleaning, whereby damage to the hair structure and/or drying out of the scalp can occur, especially if cleaning is performed frequently. Damage to the hair structure or the hair fibers, particularly split ends and/or hair breakage, can additionally be promoted by environmental influences (for example, intense solar irradiation), mechanical loads (for example, combing while blow-drying heat is applied), and chemical influences (for example, dyeing, shaping, or smoothing of the hair).

Numerous hair care agents, such as hair masks, conditioners, or hair care lotions, that can be used successfully to treat split ends and/or hair breakage are known from the prior art. However, for reasons of time and cost, hair cleaning agents that simultaneously have a care effect and a repair effect on the hair are desired by consumers for daily use.

The formulation of 2-in-1 or 3-in-1 preparations has also been known in the prior art for a long time. For example, in DE 102012201861, a conditioning hair cleaning agent that includes a cationic guar polymer, a silicone emulsion, and at least one wax in addition to an anionic surfactant is proposed. The cleaning agents having high foaming capability and give the hair improved combability and improved optical and tactile properties after the cleaning agents have been applied. However, it has been found that these agents and comparable 2-in-1 cleaning preparations known from the prior art must be optimized further, particularly with regard to the repair of damaged hair fibers and the strengthening of the hair structure.

It is therefore desirable to provide mild, care-providing hair cleaning agents that are safe for skin and hair, are highly effective against hair damage, and optimally strengthen the hair structure.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic cleaning agent includes in a cosmetic vehicle, at least one anionic and/or amphoteric (zwitterionic) surfactant, at least one cationic polysaccharide, at least one silicone, at least one wax, and at least one virgin oil and/or at least one cationic protein hydrolysate.

A method for cleaning hair and/or for eliminating, reducing, and/or avoiding hair breakage includes applying to preferably wet hair a cosmetic agent that includes at least one anionic and/or amphoteric (zwitterionic) surfactant, at least one cationic polysaccharide, at least one silicone, at least one wax, and at least one virgin oil and/or at least one cationic protein hydrolysate. The agent is rinsed out with water after an exposure time of 5 seconds to 5 minutes.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that the above-discussed problems can be solved by means of a specific mixture of at least one surfactant and specific care substances. The corresponding cleaning agents are very safe for skin and hair, care for and clean the hair, strengthen the hair structure, and significantly reduce hair damage, particularly hair breakage.

The invention relates first to a cosmetic cleaning agent that includes, in a cosmetic vehicle,

a) at least one anionic and/or amphoteric (zwitterionic) surfactant,

b) at least one cationic polysaccharide,

c) at least one silicone,

d) at least one wax, and

e) at least one virgin oil and/or at least one cationic protein hydrolysate.

In the context of the invention, a cosmetically acceptable vehicle is preferably understood to be an aqueous or aqueous-alcoholic vehicle. Especially preferably, the cosmetic vehicle includes at least 50 wt. %, more preferably at least 60 wt. %, and especially preferably at least 65 wt. %, of water. Furthermore, the cosmetic vehicle can include 0.01 to 40 wt. %, preferably 0.05 to 30 wt. %, and particularly 0.1 to 20 wt. %, of at least one alcohol, which can be selected from ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol, or mixtures of these alcohols. The water-soluble alcohols are especially preferred. Ethanol, ethyl diglycol, 1-propanol, 2-propanol, isopropanol, 1,2-propylene glycol, glycerol, benzyl alcohol, and/or phenoxyethanol and mixtures of these alcohols are particularly preferred.

The suitable anionic surfactants that can be used in the cleaning agents according to the invention include, for example:
linear and branched fatty acids having 8 to 30 C atoms (soaps),
ether carboxylic acids of the formula R—O—(CH₂—CH₂O)ₓ—CH₂—COOH, in which R is a linear or branched, saturated or unsaturated alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and/or dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
alpha-olefin sulfonates having 8 to 24 C atoms,
alkyl sulfates and/or alkyl polyglycol ether sulfate salts of the formula R—O—(CH₂—CH₂O)ₙ—CH₂—CH₂—O—SO₃X, in which R preferably means a linear or branched, saturated or unsaturated alkyl group having 8 to 30 C atoms, n means the number 0 or 1 to 12, and X means an alkali metal ion, alkaline-earth metal ion, ammonium ion, or alkanolamine ion.
sulfonates of unsaturated fatty acids having 8 to 24 C atoms and 1 to 6 double bonds,
esters of tartaric acid and citric acid with alcohols that are products of the attachment of approximately 2-15 molecules of ethylene oxide and/or propylene oxide to fatty alcohols having 8 to 22 C atoms, and/or
alkyl and/or alkenyl ether phosphates of the formula

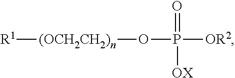

in which R¹ preferably stands for an aliphatic hydrocarbon group having 8 to 30 carbon atoms, R² stands for hydrogen, a group (CH₂CH₂O)ₙR¹, or X, n stands for numbers from 0 to 10, and X stands for hydrogen, an alkali metal or alkaline-earth metal, or the group —NR³R⁴R⁵R⁶, with R³ to R⁶ standing, independently of each other, for a C₁ to C₄ hydrocarbon group.

Preferred anionic surfactants are alkyl sulfates and/or alkyl polyglycol ether sulfate salts of the formula R—O—(CH₂—CH₂O)ₙ—CH₂—CH₂—O—SO₃X, in which R preferably stands for a straight-chain or branched, saturated or mono- or polyunsaturated alkyl group or alkenyl group having 8 to 24 carbon atoms, n stands for 0 or 1 to 12, and X stands for an alkali metal or alkaline-earth metal or for triethanolamine. Especially preferred anionic surfactants are straight-chain or branched alkyl polyglycol ether sulfate salts of the previously stated formula that include an alkyl group having 8 to 18 and particularly 10 to 16 C atoms and include 1 to 6 and particularly 2 to 4 ethylene oxide units. The sodium, magnesium, and/or triethanolamine salts of linear or branched lauryl, tridecyl, and/or myristyl sulfates that have a degree of ethoxylation of 2 to 4 are very especially preferred. The cleaning agents according to the invention particularly preferably include at least one anionic surfactant having the INCI name Sodium Laureth Sulfate, because these surfactants have an especially even balance between foaming capability and mildness.

The proportion of the one or more surfactants by weight with respect to the total weight of the compositions is preferably 0.5 to 20 wt. %, more preferably 1 to 15 wt. %, especially preferably 2 to 12.5 wt. %, and particularly 3 to 10 wt. %.

The suitable amphoteric and/or zwitterionic surfactants that can be used in the cleaning agents according to the invention include, for example, one or more compounds of the following formulas (I) to (VII), in which the group R stands for a straight-chain or branched, saturated or mono- or polyunsaturated alkyl or alkenyl group having 7 to 23 carbon atoms (formulas (I) and (II)) or for a straight-chain or branched, saturated or mono- or polyunsaturated alkyl or alkenyl group having 8 to 24 carbon atoms (formulas (III) to (VII)):

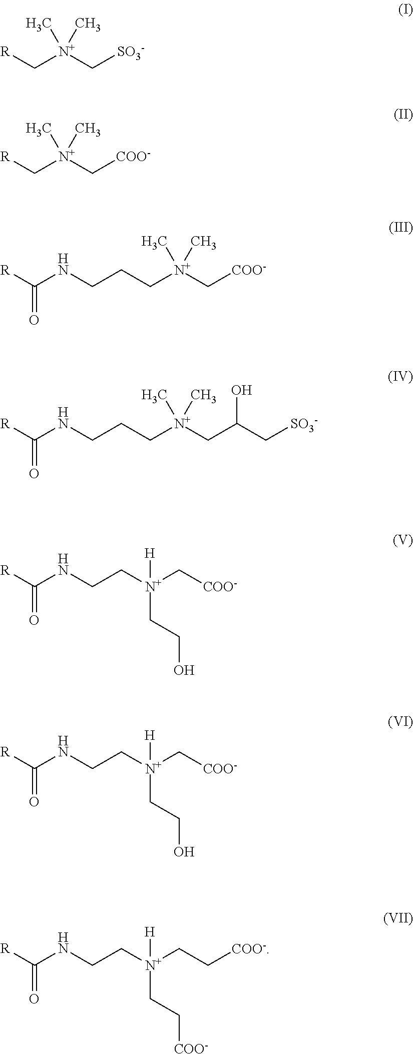

Preferred amphoteric and/or zwitterionic surfactants of one of the previously stated formulas (I) to (VII) include, as group R, predominantly a straight-chain or branched, saturated or mono- or polyunsaturated alkyl group having 8 to 20, more preferably 8 to 18, and particularly 8 to 16, C atoms. Amphoteric and/or zwitterionic surfactants in the case of which the group R is derived from coconut fat are especially preferred.

The amphoteric/zwitterionic surfactants known under the INCI names Sodium Cocoamphoacetate, Disodium Cocoamphodiacetate, Sodium Lauroamphoacetate, Sodium Lauroamphodiacetate, Sodium Cocoamphopropionate, Disodium Cocoamphodipropionate, Coco Betaine, Lauryl Betaine, Cocamidopropyl Betaine, and/or Lauramidopropyl Betaine, which are commercially available from several providers, are very especially preferred. Surfactants with the INCI names Cocoampho(di)acetate and/or Cocamidopropyl Betaine are particularly preferred.

The proportion by weight of the one or more amphoteric and/or zwitterionic surfactants with respect to the total weight of the compositions is preferably 0.5 to 20 wt. %, more preferably 1 to 15 wt. %, especially preferably 1.25 to 10 wt. %, and particularly 1.5 to 7.5 wt. %.

In the context of the invention, it was worthwhile to find an especially mild surfactant base that is gentle on the scalp for the cleaning agents in order not to stress the hair. A specific mixture of anionic and amphoteric surfactants has proven to be especially suitable for this purpose.

In a first preferred embodiment, cleaning agents according to the invention therefore preferably include
ai) at least one anionic surfactant from the group of the alkyl sulfates and/or alkyl polyglycol ether sulfates of the formula R—O—(CH₂—CH₂O)ₙ—CH₂—CH₂—O—SO₃X, in which R preferably stands for a straight-chain or branched, saturated or mono- or polyunsaturated alkyl group or alkenyl group having 8 to 24 carbon atoms, n stands for 0 or 1 to 12, and X stands for an alkali metal or alkaline-earth metal or for triethanolamine, wherein anionic surfactants having the INCI name Sodium Laureth Sulfate are especially preferred, and
aii) at least one amphoteric (zwitterionic) surfactant from the group of the (C₈-C₂₄) alkyl ampho(di)acetates and/or -propionates and/or from the group of the (C₈-C₂₄) alkyl amido (C₁-C₄) alkyl betaines, wherein amphoteric (zwitterionic) surfactants having the INCI names Cocoampho(di)acetate and/or Cocamidopropyl Betaine are especially preferred.

In this embodiment, it is especially preferred if the cosmetic cleaning agents according to the invention include
ai) at least one anionic surfactant having the INCI name Sodium Laureth Sulfate,
aii) at least one amphoteric (zwitterionic) surfactant having the INCI name Cocoampho(di)acetate, and
aiii) at least one amphoteric (zwitterionic) surfactant having the INCI name Cocamidopropyl Betaine.

Particularly preferred cleaning agents in this embodiment include the surfactants ai), aii), and aiii) in a weight ratio (ai):(aii)+aiii))) in the range of 5:1 to 1:2, preferably 4.5:1 to 1:1, especially preferably 4.25:1 to 1.5:1, and particularly 4:1 to 2:1.

A second essential constituent of the cosmetic cleaning agents according to the invention is a cationic polysaccharide, the proportion of which by weight with respect to the total weight of the composition is preferably 0.01 to 5 wt. %, more preferably 0.05 to 3 wt. %, especially preferably 0.1 to 2 wt. %, and particularly 0.2 to 1 wt. %.

Cationic polysaccharides preferred according to the invention are
quaternized cellulose derivatives, which are commercially available under the names Celquat® and Polymer JR®; the compounds Celquat® H 100, Celquat® L 200, and Polymer JR® 400 are preferred quaternized cellulose derivatives,
hydrophobically modified cellulose derivatives, such as the cationic polymers sold under the trade name SoftCat®,
cationic alkyl polyglycosides,
cationized honey, such as the commercial product Honeyquat® 50,
cationic guar derivatives, such as in particular the products sold under the trade names Cosmedia® Guar, N-Hance®, and Jaguar®.

Especially preferred cationic polysaccharides are the cationic hydroxyalkyl guar derivatives, preferably cationic hydroxyethyl trimethylammonium guar and/or cationic hydroxypropyl trimethylammonium guar having average molecular weights between 100,000 and 2,000,000 daltons. The cationic guar polymers known under the INCI name Guar Hydroxypropyltrimonium Chloride and having a molecular weight (weight average) between 200,000 and 1,600,000 daltons are particularly preferred. The cationic charge density of these guar polymers is preferably at least 0.4 mEq/g, preferably at least 0.5 mEq/g, and particularly at least 0.6 mEq/g. Their nitrogen content preferably lies in the range of 1.1 to 1.8 wt. % (with respect to their total weight). In summary, cosmetic compositions according to the invention that include a cationic guar derivative as a cationic polysaccharide are preferred.

Accordingly, cleaning agents preferred according to the invention include at least one cationic polysaccharide, which is selected from quaternized cellulose derivatives, which can be hydrophobically modified, cationic alkyl polyglycosides, cationized honey, and/or cationic guar derivatives, wherein cationic guar derivatives, particularly a guar derivative known under the INCI name Guar Hydroxypropyltrimonium Chloride, are especially preferred.

A third essential constituent of the cosmetic cleaning agents according to the invention is a silicone, the proportion of which by weight with respect to the total weight of the composition is preferably 0.01 to 5 wt. %, more preferably 0.05 to 3 wt. %, especially preferably 0.1 to 2 wt. %, and particularly 0.2 to 1 wt. %.

Suitable silicones that can be preferably used in the cleaning agents according to the invention are
(i) polyalkylsiloxanes, polyarylsiloxanes, polyalkylarylsiloxanes, which can be volatile or non-volatile, straight-chained, branched, or cyclic, cross-linked or uncross-linked;
(ii) polysiloxanes that include in their general structure one or more organofunctional groups, which can be selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacyl amino groups;
   k) carboxy groups;
   l) sulfonic acid groups; and
   m) sulfate groups or thiosulfate groups;
(iii) linear polysiloxane (A)/polyoxyalkylene (B) block copolymers of the type (A-B)ₙ with n>3;
(iv) graft silicone polymers having an organic backbone that does not include silicone, which consist of an organic main chain, which is formed of organic monomers that do not include silicone, on which main chain at least one polysiloxane macromonomer has been grafted in the chain and optionally at at least one chain end;
(v) graft silicone polymers having a polysiloxane backbone, onto which organic monomers that do not include silicone have been grafted, which graft silicone polymers have a polysiloxane main chain, on which at least one organic macromonomer that does not include silicone has been grafted in the chain and optionally at at least one of the ends of the main chain;
(vi) or mixtures thereof

Especially preferred silicones are water-insoluble, non-volatile polydialkyl-, polyalkylaryl-, and/or polydriarylsiloxanes, wherein water-insoluble, non-volatile polydialkylsiloxanes are especially preferred. Suitable alkyl groups should preferably be understood to include straight-chain or branched C₁-C₄ alkyl groups, particularly methyl groups. Very especially preferred silicones are polydimethylsiloxanes that preferably have a viscosity in the range of 1,000 to 1,000,000 cSt, more preferably 5,000 to 500,000 cSt, especially preferably 10,000 to 200,000 cSt, and particularly 30,000 to 100,000 cSt, at a temperature of 25° C. The viscosity of the polysiloxanes can be measured, for example, at 25° C. by means of a glass capillary viscometer in accordance with the Dow Corning Corporate Test Method CTM0004 (20 Jul. 1970).

In another preferred embodiment, cosmetic cleaning agents according to the invention are accordingly characterized in that they include at least one silicone that is selected from water-insoluble, non-volatile polydialkyl-, polyalkylaryl-, and/or polydriarylsiloxanes, preferably from polydimethylsiloxanes that have a viscosity in the range of 1,000 to 1,000,000 cSt, preferably 5,000 to 500,000 cSt, especially preferably 10,000 to 200,000 cSt, and particularly 30,000 to 100,000 cSt, at 25° C.

A fourth essential constituent of the cosmetic cleaning agents according to the invention is a wax, the proportion of which by weight with respect to the total weight of the composition is preferably 0.01 to 2 wt. %, more preferably 0.05 to 1 wt. %, especially preferably 0.075 to 0.75 wt. %, and particularly 0.1 to 0.5 wt. %.

In the context of the invention, suitable “waxes” should preferably be understood to include natural and synthetic substances that typically have the following properties: kneadable at 20° C., solid to brittle, coarse to finely crystalline, translucent to opaque, but not glassy, melting without decomposition above 40° C., of relatively low viscosity and not stringy already at a little above the melting point, highly temperature-dependent consistency and solubility, polishable under light pressure.

Natural waxes should preferably be understood to include plant waxes such as carnauba wax, candelilla wax, and/or jojoba oil and animal waxes such as beeswax, wool wax, spermaceti, and/or preen oil. Synthetic waxes should preferably be understood to include mineral waxes such as hard paraffin, ceresin, ozokerite, ester waxes such polyethylene glycol waxes or polyethylene glycol ester waxes, and/or hardened plant oils.

Especially preferred are chemically modified (particularly hardened) or unmodified waxes of plant origin, which preferably have melting points in the range of 80 to 90° C., more preferably 82 to 90° C., and particularly 85 to 88° C. Particularly preferred are hardened plant oils, particularly preferably hardened castor oil, which is commercially available from various providers under the INCI name Hydrogenated Castor Oil.

In another preferred embodiment, cosmetic cleaning agents according to the invention are accordingly characterized in that they include at least one wax, which has a melting point in the range of 80 to 90° C., wherein waxes of plant origin are preferred and hardened castor oil is especially preferred.

A fourth essential constituent of the cosmetic cleaning agents according to the invention is at least one virgin oil and/or at least one cationic protein hydrolysate. The proportion of the virgin oil by weight with respect to the total weight of the composition is preferably 0.01 to 3 wt. %, more preferably 0.05 to 2 wt. %. The proportion of the cationic protein hydrolystate by weight with respect to the total weight of the composition is preferably 0.01 to 3 wt. %, more preferably 0.05 to 2 wt. %.

Preferably triglycerides and mixtures of triglycerides are used as virgin or natural (plant) oils. Preferred natural oils are coconut oil, (sweet) almond oil, walnut oil, peach kernel oil, apricot kernel oil, avocado oil, tea tree oil, soy oil, sesame oil, sunflower oil, tsubaki oil, evening primrose oil, rice bran oil, palm kernel oil, mango kernel oil, meadowfoam seed oil, macadamia nut oil, grape seed oil, amaranth seed oil, argan oil, marula oil, bamboo oil, olive oil, wheat germ oil, pumpkin seed oil, mallow oil, hazelnut oil, safflower (seed) oil, canola oil, sasanqua oil, jojoba oil, rambutan oil, cocoa butter, and shea butter. Especially preferred are cleaning agents according to the invention that include apricot kernel oil, argan oil, manila oil, jojoba oil, and/or almond oil as a virgin oil. Apricot kernel oil, marula oil, and jojoba oil are particularly preferred.

The virgin oil is present in the cosmetic cleaning agents preferably in dispersed form, wherein the drop size of the dispersed oil is preferably 0.1 to 5 μm, especially preferably 0.5 to 3 μm.

Suitable cationic protein hydrolysates are preferably understood to include compounds of the following formula (I),

R′—X—R″ (I),

in which
R′ stands for a straight-chain or branched, saturated or unsaturated hydrocarbon group having 11 to 24 carbon atoms,
R″ means a protein, a peptide, or a hydrolysate from keratin,
X stands for —C(O)O— or —N⁺(R^{III}₂)R^{IV}— or —N(R^{III})R^{IV}— or —C(O)—N(R^{V})R^{VI}—,
R^{III} means —(CH₂)ₓ—CH₃ with x=0-22, and
R^{IV} means —CH₂—CH(OH)—CH₂— or —(CH₂)ₓ— with x=0-22;
R^{V} and R^{VI} stand independently of each other for —H or —(CH₂)ₓ—CH₃ with x=0-22.

Especially preferred are compounds of the previously stated formula (I) in which
X stands for —N⁺(CH₃)₂—CH₂—CH(OH)—CH₂— or for —C(O)—O—,
R″ stands for a keratin hydrolysate, and
R′ stands for —(CH₂)₁₇—CH₃.

Particularly preferred are cationic keratin hydrolysates of the previously stated formula (I) that have an average molar weight (weight average) in the range of 500 to 10000 Da, preferably 750 to 5000 Da, especially preferably 750 to 2000 Da, and particularly 800 to 1500 Da. It is furthermore preferred if the group R″ stands for cationic keratin hydrolysates that do not include methionine, and it is especially preferred if the R″ stands for cationic keratin hydrolysates that include large amounts of cysteine and/or cystine.

Examples of especially preferred commercial products are the products Croquat® WKP and Promois® WK-HCAQ, which are available under the INCI name Cocodimonium Hydroxypropyl Hydrolyzed Keratin.

Particularly preferred cationic protein hydrolysates according the previously stated formula are the compounds known under the INCI name Cocodimonium Hydroxypropyl Hydrolyzed Keratin.

In another preferred embodiment, cosmetic cleaning agents according to the invention are accordingly characterized in that they include at least one cationic protein hydrolysate, which is selected from compounds of the general formula (I),

R′—X—R″ (I),

in which
R′ stands for a straight-chain or branched, saturated or unsaturated hydrocarbon group having 8 to 24 carbon atoms,
R″ means a protein or a protein hydrolysate,
X stands for —C(O)O— or —N⁺(R^{III}₂)R^{IV}— or —N(R^{III})R^{IV}— or —C(O)—N(R^{V})R^{VI}—,
R^{III} means —(CH₂)ₓ—CH₃ with x=0-22,
R^{IV} means —CH₂—CH(OH)—CH₂— or —(CH₂)ₓ— with x=0-22, and
R^{V} and R^{VI} stand independently of each other for —H or —(CH₂)ₓ—CH₃ with x=0-22,
wherein especially preferred are cationic protein hydrolysates according to formula (I) in which
X stands for —N⁺(CH₃)₂—CH₂—CH(OH)—CH₂— or for —C(O)—O—,
R″ stands for a keratin hydrolysate, and
R′ stands for —(CH₂)₁₇—CH₃, and
wherein a cationic protein hydrolysate known under the INCI name Cocodimonium Hydroxypropyl Hydrolyzed Keratin is particularly preferred.

It has been found that the hair structure can be strengthened to a significant extent and that split ends and hair breakage can be significantly reduced if the cleaning agents according to the invention include a combination of virgin oil and a cationic protein hydrolysate in addition to the specific surfactants, the cationic polysaccharide, the silicone, and the wax.

Accordingly, in a second especially preferred embodiment, cosmetic cleaning agents according to the invention preferably include at least one virgin oil and at least one cationic protein hydrolysate according to the previously stated formula (I).

If the virgin oil and the cationic protein hydrolysate are used in a certain weight ratio, the care effect of the cleaning agents according to the invention is especially high and it was possible to significantly reduce and/or eliminate the hair breakage in particular.

Therefore, such cleaning agents according to the invention that include at least one virgin oil (O) and at least one cationic protein hydrolysate (P) in a weight ratio O:P of 5:1 to 1:5, more preferably 3:1 to 1:3, and particularly 2:1 to 1:2, are especially preferred in this second embodiment.

Particularly preferred cleaning agents according to the invention in this embodiment include
cationic protein hydrolysates according to formula (I) (P) in which
   X stands for —N⁺(CH₃)₂—CH₂—CH(OH)—CH₂— or for —C(O)—O—,
   R″ stands for a keratin hydrolysate, and
   R′ stands for —(CH₂)₁₇—CH₃,
especially preferably a cationic protein hydrolysate according to formula (I) known under the INCI name Cocodimonium Hydroxypropyl Hydrolyzed Keratin, and
at least one virgin oil (O) selected from apricot kernel oil, argan oil, manila oil, jojoba oil, and/or almond oil, especially preferably apricot kernel oil, manila oil, and/or jojoba oil, and particularly marula oil,
   in a weight ratio O:P of 3:1 to 1:3, preferably 2:1 to 1:2, and particularly 1.5:1 to 1:1.

A goal of the present invention was to provide cosmetic cleaning agents that have an optimal balance between cleaning that is thorough yet gentle on the hair and the scalp and excellent care. “Care” is taken to mean in particular the strengthening of the hair structure and the elimination, reduction, or prevention of split ends or hair breakage, particularly of hair breakage. It was determined that an optimal balance between cleaning and care can be achieved if the cleaning agents according to the invention include the essential ingredients substantially in typical amounts. However, it was possible to achieve especially good results if the cleaning agents according to the invention include the essential ingredients a) to e) in restricted amount ranges.

In a third preferred embodiment, cosmetic cleaning agents preferred according to the invention are accordingly characterized in that
a) the proportion of the anionic and/or amphoteric (zwitterionic) surfactant by weight with respect to the total weight of the composition is 0.5 to 20 wt. %, preferably 1 to 15 wt. %, in each case,
b) the proportion of the cationic polysaccharide by weight with respect to the total weight of the composition is 0.01 to 5 wt. %, preferably 0.05 to 3 wt. %,
c) the proportion of the silicone by weight with respect to the total weight of the composition is 0.01 to 5 wt. %, preferably 0.05 to 3 wt. %,
d) the proportion of the wax by weight with respect to the total weight of the composition is 0.01 to 2 wt. %, preferably 0.05 to 1 wt. %,
e) the proportion of the virgin oil by weight with respect to the total weight of the composition is 0.01 to 3 wt. %, preferably 0.05 to 2 wt. %, and
f) the proportion of the cationic protein hydrolysate by weight with respect to the total weight of the composition is 0.01 to 3 wt. %, preferably 0.05 to 2 wt. %.

Especially preferred in this embodiment are such cleaning agents according to the invention that include
a) 0.5 to 20 wt. %, more preferably 1 to 15 wt. %, of alkyl sulfates and/or alkyl polyglycol ether sulfate salts of the formula R—O—(CH₂—CH₂O)ₙ—CH₂—CH₂—O—SO₃X, in which R preferably stands for a straight-chain or branched, saturated or mono- or polyunsaturated alkyl group or alkenyl group having 8 to 24 carbon atoms, n stands for 0 or 1 to 12, and X stands for an alkali metal or alkaline-earth metal or for triethanolamine,
b) 0.5 to 20 wt. %, more preferably 1 to 15 wt. %, of at least one of the amphoteric/zwitterionic surfactants known under the INCI names Sodium Cocoamphoacetate, Disodium Cocoamphodiacetate, Sodium Lauroamphoacetate, Sodium Lauroamphodiacetate, Sodium Cocoamphopropionate, Disodium Cocoamphodipropionate, Coco Betaine, Lauryl Betaine, Cocamidopropyl Betaine, and/or Lauramidopropyl Betaine,
c) 0.01 to 5 wt. %, more preferably 0.05 to 3 wt. %, of at least one cationic hydroxyalkyl guar derivative,
d) 0.01 to 5 wt. %, more preferably 0.05 to 3 wt. %, of at least one polydialkylsiloxane, especially preferably one polydimethylsiloxane,
e) 0.01 to 2 wt. %, more preferably 0.05 to 1 wt. %, of at least one wax of plant origin that has a melting point in the range of 80 to 90° C.,
f) 0.01 to 3 wt. %, more preferably 0.05 to 2 wt. %, of at least one virgin oil, and
g) 0.01 to 3 wt. %, more preferably 0.05 to 2 wt. %, of at least one cationic protein hydrolysate according to formula (I),
   wherein each amount specification relates to the proportion of the particular component or components by weight with respect to the total weight of the compositions.

Particularly preferred in this embodiment are such cleaning agents according to the invention that include
a) 0.5 to 20 wt. %, more preferably 1 to 15 wt. %, of at least one anionic surfactant having the INCI name Sodium Laureth Sulfate,
b) 0.5 to 20 wt. %, more preferably 1 to 15 wt. %, of at least one of the amphoteric/zwitterionic surfactants known under the INCI names Sodium Cocoamphoacetate, Disodium Cocoamphodiacetate, and/or Cocamidopropyl Betaine,
c) 0.01 to 5 wt. %, more preferably 0.05 to 3 wt. %, of at least one cationic polysaccharide known under the INCI name Guar Hydroxypropyltrimonium Chloride,
d) 0.01 to 5 wt. %, more preferably 0.05 to 3 wt. %, of at least one polydimethylsiloxane that has a viscosity in the range of 1,000 to 1,000,000 cSt, preferably 5,000 to 500,000 cSt, especially preferably 10,000 to 200,000 cSt, and particularly 30,000 to 100,000 cSt, at 25° C.,
e) 0.01 to 2 wt. %, more preferably 0.05 to 1 wt. %, of hardened castor oil (INCI name: Hydrogenated Castor Oil),
f) 0.01 to 3 wt. %, more preferably 0.05 to 2 wt. %, of apricot kernel oil, argan oil, marula oil, jojoba oil, and/or almond oil, and
g) 0.01 to 3 wt. %, more preferably 0.05 to 2 wt. %, of at least one cationic protein hydrolysate according to formula (I) known under the INCI name Cocodimonium Hydroxypropyl Hydrolyzed Keratin,
   wherein each amount specification relates to the proportion of the particular component or components by weight with respect to the total weight of the compositions.

In addition to the previously mentioned active substances and vehicle substances, the cosmetic cleaning agents according to the invention can also include a series of further ingredients that give them advantageous properties. The preferred optional active substances that can be used in the compositions according to the invention include, for example:
nonionic surfactants and/or nonionic emulsifiers, which can be used in the cosmetic cleaning agents preferably in an amount of 0.1 to 10 wt. %, more preferably 0.25 to 7.5 wt. %, and particularly 0.5 to 5 wt. % (with respect to the total weight of the cleaning agents),
further oil, wax, and/or fat components, which can be used in the cosmetic cleaning agents preferably in an amount of 0.01-10 wt. %, especially preferably 0.05-7.5 wt. %, and particularly 0.1-5 wt. % (with respect to the total weight of the cleaning agents),
anti-dandruff active substances, which can be used in the cosmetic cleaning agents preferably in an amount of 0.01 to 7.5 wt. %, especially preferably 0.05 to 5 wt. %, and particularly 0.1 to 3 wt. % (with respect to the total weight of the cleaning agents).

The suitable nonionic surfactants/emulsifiers include, for example:
C₈-C₃₀ fatty acid mono- and diesters of products of the attachment of 1 to 30 mol of ethylene oxide to glycerol,
amine oxides,
products of the attachment of 2 to 50 mol of ethylene oxide and/or 0 to 5 mol of propylene oxide to linear and branched fatty alcohols having 8 to 30 C atoms, to fatty acids having 8 to 30 C atoms, and to alkylphenols having 8 to 15 C atoms in the alkyl group,
sorbitan fatty acid esters and products of the attachment of ethylene oxide to sorbitan fatty acid esters, such as polysorbates,
sugar fatty acid esters and products of the attachment of ethylene oxide to sugar fatty acid esters,
fatty acid alkanolamides, particularly C₈-C₂₄ fatty acid mono-(C₂-C₄) alkanolamides and C₈-C₂₄ fatty acid di-(C₂-C₄) alkanolamides,
products of the attachment of ethylene oxide to fatty amines, and/or
alkyl polyglucosides.

Especially suitable nonionic surfactants are alkyl oligoglucosides, particularly alkyl oligoglucosides based on hardened C_{12/14} coconut alcohol having a DP of 1-3, which are commercially available for example under the INCI name “Coco-Glucoside,” “Decyl Glucoside,” and/or “Lauryl Glucoside,” and fatty acid alkanolamides, preferably C₈-C₂₄ fatty acid mono-(C₂-C₄) alkanolamides and particularly the compounds known under the INCI names Cocamide MEA, Lauramide MEA, Palmitamide MEA, Cocamide MIPA, and Lauramide MIPA. Further especially preferred nonionic surfactants are the C₈-C₃₀ fatty acid mono- and diesters of products of the attachment of 1 to 30 mol of ethylene oxide to glycerol. The C₁₀-C₁₆ fatty acid mono- and diesters of products of the attachment of 1 to 10 mol of ethylene oxide to glycerol are especially preferred. The compound known under the INCI name PEG-7 Glyceryl Cocoate is particularly preferred.

In another especially preferred embodiment, the cleaning agents according to the invention include, in addition to active substances a) to e), at least one nonionic surfactant, selected from alkyl polyglycosides, C₈-C₂₄ fatty acid mono-(C₂-C₄) alkanolamides, and/or C₁₀-C₁₆ fatty acid mono- and diesters of products of the attachment of 1 to 10 mol of ethylene oxide to glycerol.

Suitable further oil components and/or fat components can preferably be selected from mineral oil components and/or fatty substances.

In particular, mineral oils, paraffin oils, isoparaffin oils, and synthetic hydrocarbons are used as mineral oils. An example of a usable hydrocarbon is 1,3-di(2-ethylhexyl)cyclohexane (Cetiol® S), which is available as a commercial product. Furthermore, a dialkyl ether can be used as an oil component. Usable dialkyl ethers are, in particular, di-n-alkyl ethers having a total of 12 to 36 C atoms, particularly 12 to 24 C atoms, such as di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether, and n-hexyl n-undecyl ether, and di-tert-butyl ether, diisopentyl ether, di-3-ethyldecyl ether, tert-butyl n-octyl ether, isopentyl n-octyl ether, and 2-methylpentyl n-octyl ether. Di-n-octyl ether, which is commercially available under the name Cetiol® OE, is especially preferred.

Fatty substances should be understood to include fatty acids, fatty alcohols, and natural and synthetic waxes, which can be present both in solid form and in liquid form in aqueous dispersion. Linear and/or branched, saturated and/or unsaturated fatty acids having 6-30 carbon atoms can be used as fatty acids. Fatty acids having 10-22 carbon atoms are preferred. Among these, the isostearic acids, such as the commercial products Emersol® 871 and Emersol® 875, isopalmitic acids, such as the commercial product Edenor® IP 95, and all further fatty acids sold under the Edenor® commercial designations (Cognis) should be mentioned as examples. Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, erucic acid, and their technical mixtures. The fatty acid cuts that can be obtained from coconut oil or palm oil are typically especially preferred. The use of stearic acid is generally particularly preferred.

Saturated or mono- or polyunsaturated, branched or unbranched fatty alcohols having C₆-C₃₀, preferably C₁₀-C₂₂, and very especially preferably C₁₂-C₂₂ carbon atoms can be used as fatty alcohols. For example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol, and behenyl alcohol, and their Guerbet alcohols, can be used, wherein this list should provide examples and should not be restrictive. However, the fatty alcohols are derived from preferably natural fatty acids, wherein it can typically be assumed that the fatty alcohols are obtained from the esters of the fatty acids by reduction. Fatty alcohol cuts that are produced by reducing naturally occurring triglycerides such as beef tallow, palm oil, peanut oil, colza oil, cottonseed oil, soy oil, sunflower oil, and linseed oil or fatty acid esters arising from the products of the transesterification of said triglycerides with corresponding alcohols, and thus are a mixture of different fatty alcohols, are also usable according to the invention. Such substances can be purchased, for example, under the names Stenol®, e.g., Stenol® 1618, Lanette®, e.g., Lanette® O, Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16, or Isocarb® 24. Of course, wool-wax alcohols, which can be purchased, for example, under the names Coronoa®, White Swan®, Coronet®, or Fluilan®, can also be used according to the invention. Solid paraffins or isoparaffins, carnauba waxes, beeswaxes, candelilla waxes, ozokerites, ceresin, spermaceti, sunflower wax, fruit waxes such as apple wax or citrus wax, and microcrystalline waxes from PE or PP can be used as natural or synthetic waxes. Such waxes are available, for example, through Kahl & Col., Trittau.

Further solids are, for example:
ester oils. Ester oils should be understood include the esters of C₆-C₃₀ fatty acids with C₂-C₃₀ fatty alcohols. The monoesters of the fatty acids with alcohols having 2 to 24 C atoms are preferred. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, and their technical mixtures. Examples of the fatty alcohol components in the ester oils are isopropyl alcohol, 1-hexanol, capryl alcohol, 2-ethylhexanol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol, and brassidyl alcohol and their technical mixtures. Isopropyl myristate (Rilanit® IPM), isononaoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V) are especially preferred.
dicarboxylic acid esters, such as di-n-butyl adipate, di(2-ethylhexyl) adipate, di(2-ethylhexyl) succinate, and diisotridecyl acelaate, and diol esters, such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate,
symmetric, asymmetric, or cyclic esters of carbonic acid with fatty alcohols,
glycerol carbonate or dicaprylyl carbonate (Cetiol® CC),
ethoxylated or non-ethoxylated mono-, di-, and tri fatty acid esters of saturated and/or unsaturated linear and/or branched fatty acids with glycerol, such as Monomuls® 90-O18, Monomuls® 90-L12, Cetiol® HE, or Cutina® MD.

Suitable anti-dandruff active substances can be selected from piroctone olamine, climbazole, zinc pyrithione, ketoconazole, salicylic acid, sulfur, selenium sulfide, tar preparations, undecenoic acid derivatives, burdock root extracts, Populus extracts, nettle extracts, walnut shell extracts, birch extracts, willow bark extracts, rosemary extracts, and/or arnica extracts. Climbazole, zinc pyrithione, and piroctone olamine are especially preferred. Zinc pyrithione is particularly preferred.

The further optional components that can be used in the cleaning agents according to the invention include, for example:

vitamins, vitamin derivatives, and/or vitamin precursors,

plant extracts, and/or

humectants.

Suitable vitamins should be understood to include preferably the following vitamins, provitamins, and vitamin precursors and their derivatives:

Vitamin A: The group of the substances referred to as vitamin A includes retinol (vitamin A₁) and 3,4-didehydroretinol (vitamin A₂). β-Carotene is the provitamin of retinol. For example, vitamin A acid and esters thereof, vitamin A aldehyde, and vitamin A alcohol and esters thereof such as the palmitate and the acetate are considered as a vitamin A component.
Vitamin B: The vitamin B group or the vitamin B complex includes, among other things:

Vitamin B₁ (thiamine)

Vitamin B₂ (riboflavin)

Vitamin B₃. The compounds nicotinic acid and nicotinic acid amide (niacinamide) are often designated as vitamin B₃.

Vitamin B₅ (pantothenic acid and panthenol). Of this group, panthenol is preferably used. Usable derivatives of panthenol are, in particular, the esters and ethers of panthenol and cationically derivatized panthenols. Individual representatives are, for example, panthenol triacetate, panthenol monoethyl ether and the monoacetate thereof, and cationic panthenol derivatives.

Vitamin B₆ (pyridoxine, and pyridoxamine and pyridoxal).

Vitamin C (ascorbic acid): The use in the form of the palmitic acid ester, the glucosides, or the phosphates can be preferred. The use in combination with tocopherols can likewise be preferred.
Vitamin E (tocopherols, particularly α-tocopherol).
Vitamin F: The term “vitamin F” is typically taken to mean essential fatty acids, particularly linoleic acid, linolenic acid, and arachidonic acid.
Vitamin H: The compound (3aS,4S,6aR)-2-oxohexahydrothienol[3,4-d]imidazole-4-valeric acid is referred to as vitamin H, but the trivial name biotin has become established for this compound in the meantime.

The cosmetic cleaning agents according to the invention can preferably include vitamins, provitamins, and vitamin precursors from groups A, B, E, and H. Particularly preferred are nicotinic acid amide, biotin, pantolactone, and/or panthenol. Vitamins, vitamin derivatives, and/or vitamin precursors can be used in the cleaning agents according to the invention preferably in an amount of 0.001 to 5 wt. %, preferably 0.005 to 3 wt. %, and particularly 0.01 to 2 wt. %, in each case (with respect to the total weight of the cleaning agents).

Suitable plant extracts should be understood to include extracts that can be produced from all parts of a plant. These extracts are typically produced by extraction from the entire plant. However, in individual cases, it can also be preferred that the extracts are produced exclusively from flowers and/or leaves of the plant. Above all, the extracts from green tea, oak bark, nettles, witch hazel, hops, chamomile, burdock root, horsetail, whitethorn, linden blossoms, lychee, almond, aloe vera, spruce needle, Aesculus, sandalwood, juniper, coconut, mango, apricot, lime, wheat, kiwi, melon, orange, grapefruit, sage, rosemary, birch, mallow, meadowfoam, wild thyme, yarrow, thyme, melissa, restharrow, coltsfoot, marshmallow, ginseng, ginger root, Echinacea purpurea, Olea europea, Boerhavia diffusa roots, Foeniculum vulgaris, and Apim graveolens are suitable. Especially preferred for use in the compositions according to the invention are the extracts from green tea, nettles, witchhazel, chamomile, aloe vera, ginseng, Echinacea purpurea, Olea europea, and/or Boerhavia diffusa roots. Water, alcohols, and mixtures thereof can be used as extracting agents for producing the stated plant extracts. Among the alcohols, lower alcohols such as ethanol and isopropanol, but in particular polyhydric alcohols such as ethylene glycol and propylene glycol, are preferred, both as a sole extracting agent and in mixture with water. Plant extracts based on water/propylene glycol at a ratio of 1:10 to 10:1 have proven to be especially suitable. The plant extracts can be used both in pure form and in diluted form. Provided they are used in diluted form, they typically include approximately 2 to 80 wt. % of active matter and, as a solvent, the extracting agent or extracting agent mixture used to obtain the active matter. The plant extracts can be used in the cleaning agents according to the invention preferably in an amount of 0.01 to 10 wt. %, more preferably 0.05 to 7.5 wt. %, and particularly 0.1 to 5 wt. % (with respect to the total weight of the cleaning agents).

Suitable humectants or penetration aids and/or swelling agents that can be added to the cleaning agents according to the invention are, for example, urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, such as propylene glycol monoethyl ether, carbonates, hydrogen carbonates, diols and triols, and particularly 1,2-diols and 1,3-diols such as 1,2-propanediol, 1,2-pentanediol, 1,2-hexanediol, 1,2-dodecanediol, 1,3-propanediol, 1,6-hexanediol, 1,5-pentanediol, and 1,4-butanediol. The humectants can be used in the cleaning agents according to the invention preferably in amounts of 0.01 to 10 wt. %, more preferably in amounts of 0.05 to 5 wt. %, and particularly in amounts of 0.1 to 3 wt. % —with respect to the total weight of the cleaning agents according to the invention.

Further active substances, aids, and additives that can be used in the cleaning agents according to the invention are, for example:
UV filters,
thickeners such as gelatin or plant gum, such as agar-agar, guar gum, alginates, xanthan gum, gum arabic, gum karaya, locust bean gum, linseed gums, dextranes, cellulose derivatives, e.g., methyl cellulose, hydroxyalkyl cellulose, and carboxymethyl cellulose, starch fractions and derivatives such as amylose, amylopectin, and dextrins, clays and phyllosilicates such as bentonite or fully synthetic hydrocolloids such as polyvinyl alcohol, the Ca, Mg, or Zn soaps,
structurants such as maleic acid and lactic acid,
dyes for coloring the agent,
substances for adjusting the pH value, for example α- and β-hydroxycarboxylic acids such as citric acid, lactic acid, malic acid, glycolic acid,
active substances such as bisabolol,
complexing agents such as EDTA, NTA, β-alanine diacetic acid, and phosphonic acids,
ceramides. Ceramides are understood to include N-acylsphingosine (fatty acid amides of sphingosin) or synthetic analogs of such lipids (pseudo-ceramides),
propellants such as propane-butane mixtures, N₂O, dimethyl ether, CO₂, and air,
antioxidants,
pearlizing agents such as EGDS or PEG-3 distearate,
preservatives, such as sodium benzoate or salicylic acid,
viscosity regulators such as salts (NaCL).

The cosmetic compositions according to the invention preferably have pH values in the range of 2.5 to 6.5, preferably 3.5 to 6, and particularly 4 to 5.5.

As mentioned at the beginning, the cleaning agents according to the invention have excellent properties in use on hair. In addition to the gentle and thorough cleaning, they give hair treated therewith improved properties. Treated dry hair (particularly hair previously damaged in the structure thereof and fragile hair) has improved hair texture and greater smoothness, shine, and softness in addition to significantly less hair breakage. Treated wet hair (particularly hair previously damaged in the structure thereof and fragile hair) has fewer or no knots in addition to significantly less hair breakage. A further advantage of the cleaning agents according to the invention is that they support the resistance of the hair surface to physical and/or chemical hair damage.

Second, the present invention relates to the use of a cosmetic cleaning agent according to the invention to eliminate, reduce, and/or avoid hair breakage.

Third, the present invention relates to a method for cleaning hair and/or for eliminating, reducing, and/or avoiding hair breakage, wherein a cosmetic agent, including

a) at least one anionic and/or amphoteric (zwitterionic) surfactant,

b) at least one cationic polysaccharide,

c) at least one silicone,

d) at least one wax, and

e) at least one virgin oil and/or at least one cationic protein hydrolysate,

is applied to the preferably wet hair and is rinsed out with water after an exposure time of 5 seconds to 5 minutes.

Statements made with regard to the use according to the invention apply, mutatis mutandis, to preferred embodiments of the use according to the invention and to the method according to the invention.

The examples below should illustrate the subject matter of the invention without restricting the subject matter of the invention to said examples.

### Examples

### 1) Production of Shampoos According to the Invention (Amounts in [Wt. %])

| | Ex. 1 | Ex. 2 | Ex. 3 | Ex. 4 | Ex. 5 |
|---|---|---|---|---|---|
| Sodium Laureth | 9 | 9 | 8 | 10 | |
| Sulfate (AS) | | | | | |
| Disodium Cocoamphodi- | 0.8 | | 1 | 0.5 | 3 |
| acetate (AS) | | | | | |
| Cocoamidopropyl | 1.6 | 2.5 | 2 | 1.5 | 4.5 |
| Betaine (AS) | | | | | |
| Cocamide MEA | 0.45 | 0.5 | | 0.35 | 0.7 |
| PEG-7 Glyceryl | 0.4 | 0.6 | 0.5 | | 0.7 |
| Cocoate | | | | | |
| PEG-40 Hydrogenated | 0.3 | 0.3 | 0.3 | 0.4 | 0.4 |
| Castor Oil | | | | | |
| Guar Hydroxypropyl- | 0.3 | 0.3 | 0.2 | 0.2 | 0.4 |
| trimonium Chloride | | | | | |
| Polydimethylsiloxane | 0.4 | 0.4 | 0.3 | 0.3 | 0.5 |
| 60000 cSt | | | | | |
| Marula oil | 0.05 | 0.05 | | | 0.05 |
| Jojoba oil | | | 0.1 | | |
| Apricot kernel oil | | | | 0.1 | |
| Hydrogenated | 0.1 | 0.15 | 0.2 | 0.1 | 0.1 |
| Castor Oil | | | | | |
| Cocodimonium | 0.05 | 0.05 | 0.05 | | 0.05 |
| Hydroxypropyl | | | | | |
| Hydrolyzed Keratin | | | | | |
| Sodium benzoate | 0.5 | 0.5 | 0.5 | 0.5 | 0.5 |
| Citric acid | 0.1-1.5 | 0.1-1.5 | 0.1-1.5 | 0.1-1.5 | 0.1-1.5 |
| NaCl | 0.1-2.5 | 0.1-2.5 | 0.1-2.5 | 0.1-2.5 | 0.1-2.5 |
| Perfume | 0.5-1 | 0.5-1 | 0.5-1 | 0.5-1 | 0.5-1 |
| Water | ad 100 | ad 100 | ad 100 | ad 100 | ad 100 |
| pH value | 4.5-5 | 4.5-5 | 4.5-5 | 4.5-5 | 4.5-5 |
| | (±0.2) | (±0.2) | (±0.2) | (±0.2) | (±0.2) |
| Viscosity (Brookfield | 6000- | 6000- | 6000- | 6000- | 6000- |
| viscometer, 20° C., | 9000 | 9000 | 9000 | 9000 | 9000 |
| 20 rpm) [mPas] | | | | | |

### 2) Evaluation of Shampoos According to the Invention

Two compositions were produced. A composition according to the invention corresponded to example 1 (A) of the table provided above and a second composition not according to the invention corresponded to a commercially available care shampoo for hair that is fragile and susceptible to split ends (B), which included 0.7 wt. % of Polymer JR 400 and 0.2 wt. % of polydimethylsiloxane instead of the care complex according to the invention, consisting of Guar Hydroxypropyltrimonium Chloride, polydimethyl siloxane, manila oil, Hydrogenated Castor Oil, and Cocodimonium Hydroxypropyl Hydrolyzed Keratin. The two compositions were applied to identically pretreated (cleaned and then pre-damaged) hair strands from Kerling International (Backnang, Germany—European natural hair 7/0; lot #04/2010; N74; length: 12 cm; weight: 1+/−0.05 g) in a typical manner and then the reduction in hair breakage was determined.

The results can be found in the following table:

| | Reduction in hair |
|---|---|
| | breakage [%] |
| Shampoo according to example 1 (A) | 94 |
| Comparison shampoo (B) | 51 |

The results in the table clearly show that the cleaning agents according to the invention fight hair breakage significantly better than comparable shampoos from the prior art.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleaning agent, including, in a cosmetic vehicle,
a) at least one anionic and/or amphoteric (zwitterionic) surfactant,
b) at least one cationic polysaccharide,
c) at least one silicone,
d) at least one wax, and
e) at least one virgin oil and/or at least one cationic protein hydrolysate.

2. The cosmetic cleaning agent according to claim 1, wherein said cosmetic cleaning agent includes
ai) the at least one anionic surfactant, which is at least one surfactant selected from the group consisting of alkyl sulfates and alkyl polyglycol ether sulfates of the formula R—O—(CH₂—CH₂O)ₙ—CH₂—CH₂—O—SO₃X, in which R is a straight-chain or branched, saturated or mono- or polyunsaturated alkyl group or alkenyl group having 8 to 24 carbon atoms, n is 0 to 12, and X is an alkali metal or alkaline-earth metal or for triethanolamine, and
aii) the at least one amphoteric (zwitterionic) surfactant, which is at least one surfactant selected from the group consisting of (C₈-C₂₄) alkyl ampho(di)acetates and -propionates, and (C₈-C₂₄) alkyl amido (C₁-C₄) alkyl betaines.

3. The cosmetic cleaning agent according to claim 1, wherein the cationic polysaccharide is selected from the group consisting of quaternized cellulose derivatives that are optionally hydrophobically modified, cationic alkyl polyglycosides, cationized honey, and cationic guar derivatives.

4. The cosmetic cleaning agent according to claim 1, wherein the silicone is one or more compounds selected from the group consisting of water-insoluble, non-volatile polydialkyl-, polyalkylaryl-, and polydriarylsiloxanes that have a viscosity in the range of 1,000 to 1,000,000 cSt.

5. The cosmetic cleaning agent according to claim 1, wherein the at least one wax has a melting point in the range of 80 to 90° C.

6. The cosmetic cleaning agent according to claim 1, wherein the cationic protein hydrolysate is one or more compounds selected from the group consisting of compounds of general formula (I),
R′—X—R″ (I),
in which
R′ stands for a straight-chain or branched, saturated or unsaturated hydrocarbon group having 8 to 24 carbon atoms,
R″ means a protein or a protein hydrolysate,
X stands for —C(O)O— or —N⁺(R^{III}₂)R^{IV}— or —N(R^{III})R^{IV}— or —C(O)—N(R^{V})R^{VI}—,
R^{III} means —(CH₂)ₓ—CH₃ with x=0-22,
R^{IV} means —CH₂—CH(OH)—CH₂— or —(CH₂)ₓ— with x=0-22, and
R^{V} and R^{VI} stand independently of each other for —H or —(CH₂)ₓ—CH₃ with x=0-22.

7. The cosmetic cleaning agent according to claim 6, wherein the cosmetic cleaning agent includes at least one virgin oil and at least one cationic protein hydrolysate according to formula (I).

8. The cosmetic cleaning agent according to claim 1, wherein
a) the proportion of the anionic and/or amphoteric (zwitterionic) surfactant by weight with respect to the total weight of the composition is 0.5 to 20 wt,
b) the proportion of the cationic polysaccharide by weight with respect to the total weight of the composition is 0.01 to 5 wt. %,
c) the proportion of the silicone by weight with respect to the total weight of the composition is 0.01 to 5 wt. %,
d) the proportion of the wax by weight with respect to the total weight of the composition is 0.01 to 2 wt. %,
e) the proportion of the virgin oil by weight with respect to the total weight of the composition is 0.01 to 3 wt. %, and
f) the proportion of the cationic protein hydrolysate by weight with respect to the total weight of the composition is 0.01 to 3 wt. %.

9. A method for cleaning hair and/or for eliminating, reducing, and/or avoiding hair breakage, wherein a cosmetic agent, including
a) at least one anionic and/or amphoteric (zwitterionic) surfactant,
b) at least one cationic polysaccharide,
c) at least one silicone,
d) at least one wax, and
e) at least one virgin oil and/or at least one cationic protein hydrolysate,
is applied to the preferably wet hair and is rinsed out with water after an exposure time of 5 seconds to 5 minutes.
